(19) United States
(12) Patent Application Publication
(10) Pub. No.: US 2007/0293882 A1
(43) Pub. Date: Dec. 20, 2007

(54) PRICKING DEVICE FOR TAKING BLOOD

(76) Inventors: Herbert Harttig, Neustadt (DE); Sven Winheim, Leimen (DE); Frank Deck, Niederkirchen (DE)

Correspondence Address: BOSE MCKINNEY & EVANS LLP, 2700 FIRST INDIANA PLAZA, 135 NORTH PENNSYLVANIA STREET, INDIANAPOLIS, IN, 46204, US

(21) Appl. No.: 11/757,759

(22) Filed: Jun. 4, 2007

Related U.S. Application Data
(63) Continuation of application No. PCT/EP05/12569, filed on Nov. 24, 2005.

(30) Foreign Application Priority Data
Dec. 2, 2004 (DE) 10 2004 058 164.9

(51) Int. Cl. A61B 17/32; A61M 5/00; A61B 17/14

(52) U.S. Cl. 606/181; 604/187

(57) ABSTRACT

The present invention provides a pricking device for taking blood comprising a contact element for being placed into contact with an extraction surface, an actuating element for manual actuation of the pricking device, a pricking element which is used to perforate the extraction surface, the pricking element being arranged in the contact element in such a way that it does not protrude from the contact element before the pricking procedure is initiated, and in such a way that a defined pricking depth is obtained during the pricking procedure. The pricking device further includes a return spring for automatically retracting the pricking element into the contact element after a pricking procedure has been completed and a locking mechanism for locking the pricking device after completion of a single pricking procedure and thereby preventing additional use of the pricking device.

### RELATED APPLICATIONS

This is a continuation application of International Application PCT/EP2005/012569, which claims priority to DE 10 2004 058 164.9, filed Dec. 2, 2004, which are hereby incorporated by reference in their entirety.

### BACKGROUND

The present invention relates to a pricking device for taking blood that is locked by a locking mechanism after it has taken a single sample of blood.

Samples of body fluids, in particular blood, are taken mainly with the aim of subsequent analysis, in order to permit a diagnosis of diseases or to monitor the metabolic status of a patient. Such samples are taken by diabetics for determining the blood sugar concentration. In order to collect only small quantities of blood for diagnostic purposes, lancets are normally used by hospital staff or patients by being briefly inserted into the patients' finger pads or other parts of the body.

U.S. Pat. No. 5,527,333 discloses a disposable device for cutting a precise incision with a defined length and depth into the skin of a patient. The device comprises a hollow housing with a top surface comprising an opening, with a front surface and rear surface, and with a bottom face that contains an oblong slit intended to be placed in contact with the patient's skin. The device also comprises a trigger arranged in an opening in the top surface. A spring is mounted in an unstressed state within the housing before the device is actuated. The spring is extended when the trigger is actuated, said spring having a first end and a second end. A cutting blade, coupled to the second end of the spring, extends through the oblong slit in order to cut into the skin when the device is actuated. The device also comprises a multiplicity of constraint elements arranged in the housing and forming an open guide surface which directs the spring and the blade to make a cut along a predetermined cutting length rather than an incision.

U.S. Pat. No. 4,889,117 discloses a disposable lancet for piercing the skin of a person's finger sufficiently to obtain a small quantity of blood. The lancet comprises a rigid, slender shaft with a piercing tip. A protective retaining tube surrounds the shaft, the shaft being displaceable in the longitudinal direction in the retaining tube. A cap is connected to one end of the shaft and surrounds one end of the retaining tube. When the cap is pressed against the end of the retaining tube, the piercing tip protrudes from the other end of the retaining tube by a defined length in order to be able to pierce the skin in this position. The cap can be moved away from the end of the retaining tube with the tip then being retracted fully into the retaining tube. After the lancet has been actuated, a resilient and pretensioned projection prevents a movement of the retaining tube in its starting position and blocks the lancet against further use.

### SUMMARY OF THE INVENTION

Embodiments incorporating the present invention provide a novel and alternative pricking device which is used for taking blood and also addresses the disadvantages of the prior art. Advantages of the present invention include the following:

(1) can be used only once and prevents repeated use;

(2) avoids the risk of contamination or infection caused by accidental contact with the pricking element (for example a needle); and,

(3) reduces the pain caused by its use.

An exemplary embodiment incorporating the present invention provides a pricking device for taking blood, comprising a contact element for placing in contact with an extraction surface, an actuating element for manual actuation of the pricking device, a pricking element which is displaceable relative to the contact element, substantially perpendicular to the extraction surface, and which is used to perforate the extraction surface, the pricking element being arranged in the contact element in such a way that it does not protrude from the contact element before the pricking procedure is initiated, and in such a way that a defined pricking depth is obtained during the pricking procedure, and a return spring for automatically retracting the pricking element into the contact element after a pricking procedure has been completed. The embodiment further includes a locking mechanism for locking the pricking device after completion of a single pricking procedure. The locking mechanism is designed such that a bolt slides across a switch along a first guide surface when the pricking procedure is initiated and, when the pricking element is retracted by the return spring after completion of the pricking procedure, the bolt slides across the switch along a second guide surface into a locked position.

In the exemplary embodiment previously described, the extraction surface is the skin of a patient, such as a finger pad, arm, foot or other part of the body. The contact element is placed onto this extraction surface. The actuating element is, for example, a push-button or lever which initiates a pricking procedure when manually actuated by a patient or hospital staff. The pricking element has a tip at one end and can be moved toward and away from the extraction surface. The defined pricking depth and movement of the pricking element substantially perpendicular to the extraction surface provide the advantage that the extraction surface is perforated with minimal pain. A cutting movement and perforating the skin too deeply are both much more painful than a perforation produced with the aid of the pricking device according to exemplary embodiments. Before actuation of the actuating element of an unused pricking device, the pricking element is fully enclosed by the contact element and does not protrude past the contact element. Therefore, the risk of injury through accidental contact with the sharp pricking element is reduced.

The return spring ensures that the pricking element is retracted completely into the contact element after a pricking procedure has been completed, thereby avoiding contamination or infection through accidental contact with the used pricking element. According to exemplary embodiments of the pricking device, only one spring is needed. The spring can have a simple (regular) form, whereas in the prior art, two or more springs are often provided to trigger a perforating procedure and/or springs with a special design are provided. Therefore, the pricking device can be produced with less expense.

Before the pricking procedure is initiated, the return spring is in a substantially unstressed state. This avoids a creeping of the spring over the course of time. This is advantageous because creeping of the spring would mean that its correct function could no longer be guaranteed after a certain period of storage.

The locking mechanism locks the pricking device according to exemplary embodiments after it has been used once, thereby preventing repeated use of the pricking device. This has the advantage of avoiding contamination or infection by a used pricking element. Therefore, the pricking device is not reusable and is to be disposed of after it has been used just once.

According to an exemplary embodiment, the locking action is obtained by a bolt being guided across guide surfaces into a locked position.

Also according to an exemplary embodiment, the pricking device has a usage indicator for showing whether the pricking device has been used or is unused. This advantageously allows a user to immediately determine whether a pricking device has been used and whether the device can now be used or must be disposed of. The user can do this without having to check whether the pricking device is already locked.

The usage indicator is based on the fact that before the pricking device is actuated, the actuating element is arranged relative to the contact element differently than it is after the pricking device has been locked by the locking mechanism. For example, the actuating element or the contact element can have a marking in the form of at least one index hole, groove, or color marking which is visible only before or after actuation and which is concealed, before or after actuation, by the other respective structural part that does not comprise the marking. The actuating element can be arranged differently relative to the contact element prior to the actuation of the pricking device. In an exemplary embodiment, this is achieved because one of these two structural parts are pushed into the other or twisted relative to the other upon actuation. According to another embodiment of a usage indicator, the status of the locking mechanism is visually determinable, for example, by the position of the actuating element or the bolt. In this embodiment, parts of the pricking device can be transparent if this is necessary to visually determine the status of the locking mechanism in the interior of the pricking device.

The pricking element according to an embodiment comprises a needle, a lancet, or a blade.

According to an exemplary embodiment, the pricking device comprises a guide for the pricking element for guiding the pricking element while it is being displaced relative to the contact element. This guide ensures precise movement of the pricking element in the desired pricking direction and as a result provides that the skin can be pricked with less pain.

In the pricking device according to one embodiment, the return spring is arranged such that a compression or an expansion of the return spring takes place during the pricking procedure. By means of this compression or expansion of the return spring, the energy required for automatic retraction of the pricking element into the contact element, after completion of a pricking procedure, is stored in the spring.

In the pricking device according to an exemplary embodiment, the actuating element is a push-button with a push surface, or a slide with lateral actuating surfaces for applying a force to the actuating element in the pricking direction, or a lever which can turn about a hinge pin in order to apply a torque.

According to one embodiment of the present invention, two locking mechanisms with identical actions are arranged symmetrically with respect to the pricking element. This advantageously permits double locking the pricking device after completion of a single pricking procedure and ensures that it is locked more securely. Even if one of the two locking mechanisms is defective, repeated use of the pricking device is prevented.

In the pricking device according to another embodiment, it is also possible for two contact elements and two return springs to be arranged symmetrically with respect to the pricking element. In this embodiment, the pricking device has one actuating element engaging across these.

According to an embodiment of the present invention, the contact element is mounted displaceably in the actuating element or the actuating element is mounted displaceably in the contact element. Upon actuation of the pricking device, the two structural parts are displaced relative to one another, in particular, with one being pushed into the other.

In the pricking device according to another embodiment, the pricking element is preferably fixedly connected to the actuating element.

The pricking element can be made of metal and be encapsulated by an actuating element made of plastic. This embodiment has the advantage of being inexpensive and provides an uncomplicated production.

According to an exemplary embodiment, the bolt is fixedly connected to the contact element or the actuating element. The bolt, in the locked position, engages behind a projection or in a depression.

According to one embodiment of the pricking device, when the defined pricking depth is reached, a first limit stop contained in the actuating element engages a second limit stop contained in the contact element. This is one possibility of obtaining a defined pricking depth. The pricking element has reached its deepest excursion when the limit stop of the actuating element strikes the limit stop of the contact element.

According to an exemplary embodiment, the switch is a deflector which interacts with a catch hook provided as bolt, the deflector being fixedly connected to the contact element and the catch hook being fixedly connected to the actuating element. In this embodiment, the deflector is arranged such that before actuation and during displacement of the pricking element toward the extraction surface, it separates the catch hook from a depression in the contact element and guides the catch hook along a first guide surface. Following the completion of a pricking procedure, the catch hook is guided along a second guide surface and is hooked into the depression in the locked position.

According to an exemplary embodiment, the pricking device can have a substantially flat design and be built up in layers. For example, the contact element, the actuating element, the pricking element, the return spring and the locking mechanism can be produced from a steel strip. The steel strip can have a width of 10 mm to 50 mm, more particularly between 20 mm to 40 mm, and a thickness of 0.1 mm to 2 mm. Individual pricking devices are obtained by mechanical cutting from the steel strip and, if appropriate, from additional components connected to the latter. Other possible components include film strips. In another embodiment, the steel strip is connected on both sides to at least one film strip. The film strips may be used, for example, as an opaque covering of the pricking device, as a usage indicator, as a grip surface for holding or actuating the pricking device, for guiding the components produced from the steel strip during the actuation of the pricking device, or as protective films.

The steel strip can be connected on both sides to a plurality of mutually displaceable film strips. In such an arrangement, the film strips move relative to one another, for example, when the actuating element, together with a film connected to it, is displaced relative to the contact element and to a film connected to the latter.

In an exemplary embodiment, the one or more film strips connected to the steel strip have a thickness of 50 μm to 2 mm. The film strip can be transparent if a usage indicator arranged under it is intended to be visually observable. It is connected to the steel strip, for example, by an adhesive. In an exemplary embodiment, the adhesive contains glass beads. The glass beads, being a defined size (for example, with a diameter of 50 μm), ensure a defined play between the individual layers of the pricking device that are displaced relative to one another during actuation of the pricking device.

According to another exemplary embodiment, the actuating element is a cylindrical sleeve which is closed at the top and which has at least one lateral recess. In this embodiment, the contact element is a cylindrical sleeve with a central longitudinal bore. A return spring, designed as a helical spring, surrounds the pricking element and is supported on an inner, first annular surface of the actuating element and on an upper, second annular surface of the contact element. The bolt is at least one guide block which is fixedly connected to the contact element by a spring element and protrudes into the lateral recess of the actuating element. The switch is fixedly connected to the actuating element and is designed such that the guide block moves the switch from a rest position upon actuation of the pricking device. After the guide block has passed, the switch automatically moves back into the rest position. A second switch fixedly connected to the actuating element is designed such that the guide block moves the second switch from a starting position to a locked position as the pricking device moves. The second switch then moves back automatically into the starting position after the guide block has passed and consequently causes a locking of the guide block to the actuating element. As the second switch locks the guide block, it bears on an abutment.

According to another embodiment, the actuating element is designed as a lever that can turn about a hinge pin and the bolt is a distal end of the lever. In this embodiment, the contact element is a housing with a recess. A proximal end of the lever protrudes through the recess before the pricking device is actuated and the hinge pin is fixedly connected to the housing. The lever is secured by a holding element before the pricking device is actuated, with a release force being needed in order to release the lever. In this embodiment, the holding element is designed to break, buckle, or free the lever when the release force is exceeded. The release force is created by manual actuation of the pricking device and, after the lever is freed by the holding element, this defined force accelerates the actuating element and pricking element in the direction of the extraction surface with a defined acceleration. This avoids a tentative pricking movement and also the greater pain associated with the latter.

Before the pricking device is actuated, the lever lies with its distal end on a contact surface of the pricking element. Upon manual actuation of the proximal end of the lever, the distal end of the lever applies a force to the pricking element in the pricking direction. In this embodiment, the contact surface is delimited by an edge and the distal end of the lever slides across the contact surface to the edge. The return spring then draws the pricking element completely into the housing as soon as the distal end of the lever has passed the edge of the contact surface and no longer applies a force to the pricking element in the pricking direction. In the locked position, the distal end of the lever can be fixed between an inside wall of the housing and a second guide surface and the pricking element is retracted completely into the housing such that the lever cannot turn about the hinge pin. Also in the locked position, the proximal end of the lever is substantially positioned in the housing.

Another exemplary embodiment provides a pricking device for taking blood with a contact element being placed into contact with an extraction surface and an actuating element for manual actuation of the pricking device, the actuating element being designed as a lever which can turn about a hinge pin and interacts with a pricking element for perforating the extraction surface. The actuating element is secured by a holding element before the pricking device is actuated and a release force is needed to release the lever. In this embodiment, the extraction surface is the skin of a patient such as a finger pad, arm, foot or other part of the body. The contact element is placed onto this extraction surface. The actuating element, designed as a lever, initiates a pricking procedure when manually actuated by a patient or hospital staff. The pricking element has a tip or blade and can be moved toward and away from the extraction surface. During a pricking procedure, the pricking element perforates the patient's skin by a pricking depth defined by the structure of the pricking device.

The pricking element is designed such that it breaks, buckles, or frees the lever when the release force is exceeded. Accordingly, the release force is generated by manual actuation of the pricking device and, after the lever is freed from the holding element, this defined force accelerates the actuating element and pricking element in the direction of the extraction surface with a defined acceleration. This avoids a tentative pricking movement and also the greater pain associated with the latter.

The lever has a proximal end and a distal end. Before actuating the pricking device, the proximal end is arranged such that a force is directly or indirectly applied to it upon manual actuation. A direct force is applied, for example, when the proximal end protrudes from the contact element and the pricking device is actuated by application of pressure by a finger directly to this proximal end. The force is indirectly applied when the user exerts a force, for example, on a push-button or slide, in order to actuate the pricking device. The force is thereby transmitted from this push-button or slide to the proximal end of the lever. By means of the directly or indirectly applied force, the release force for releasing the lever is initially exerted, followed by the acceleration force for accelerating the pricking element toward the extraction surface.

According to an exemplary embodiment, before the pricking device is actuated, the distal end of the lever rests on a contact surface of a pricking element which is displaceable in a direction perpendicular to the extraction surface. By manual actuation of the proximal end of the lever, the distal end applies a force to the contact surface of the pricking element. According to another embodiment, the distal end of the lever is fixedly connected to the pricking element and the pricking element is designed as a blade.

According to an exemplary embodiment, the pricking device also comprises a locking mechanism for locking the pricking device after completion of a pricking procedure, which mechanism locks the lever in an end position. Repeated use of the pricking element, and the infection possibly associated with repeated use, is thereby avoided.

An exemplary embodiment of the pricking device has a substantially flat design and is produced at least partially from a steel strip. The contact element, the actuating element, the pricking element, the return spring and the locking mechanism are produced from the steel strip by micropunching, laser cutting, etching, or a combination thereof.

According to an exemplary method, the contact element and the actuating element are produced integrally, being held together by a holding part, and the holding part is thereby removed after the contact element and actuating element have been finished. The contact element and actuating element are rigidly connected to one another by the holding part during production, with the result that a high level of manufacturing precision can be achieved.

Component parts of a multiplicity of contiguous pricking devices are produced from the steel strip. The individual pricking devices are separated from one another by punching away the connecting elements provided between the respective devices. When the contiguous pricking devices are separated from one another, any film strips connected to the steel strip are severed by punching with straight cuts. According to one exemplary embodiment, the steel strip is connected to at least one film strip by adhesive bonding or thermal bonding. In thermal bonding, two film strips arranged on both sides of the steel strip are welded over a recess in the steel strip.

Further details and advantages of the present invention are explained hereafter on the basis of an exemplary embodiment with reference to the attached figures. The features illustrated therein can be used individually or in combination to provide exemplary embodiments of the present invention.

### BRIEF DESCRIPTION OF THE DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1A is a schematic view of a first exemplary embodiment of a pricking device;

FIG. 1B is a cross-section of line A-A in FIG. 1A illustrating the pricking device having several layers;

FIGS. 2A-C are schematic views of the pricking device shown in FIG. 1A being actuated from an initial position to a locked position;

FIG. 3 is a schematic view of a second exemplary embodiment of a pricking device;

FIGS. 4A-C are schematic views of the pricking device shown in FIG. 3 being actuated from an initial position to a locked position;

FIG. 5A is a cross-section of line VA-VA in FIG. 5B of a third exemplary embodiment of a pricking device;

FIG. 5B is a side view of the upper part of the pricking device shown in FIG. 5A;

FIG. 6 is a schematic view of a fourth exemplary embodiment of a pricking device including an actuating element designed as a lever that can turn about a hinge pin and a holding element;

FIGS. 7A-B are schematic views of the pricking device shown in FIG. 6 being actuated from an initial position to a locked position; and,

FIGS. 8A-B are schematic views of a fifth exemplary embodiment of a pricking device including a rotatable lever and blade for perforating the extraction surface being actuated from an initial position to a locked position.

Corresponding reference numerals are used to indicate corresponding parts throughout the several views.

### DETAILED DESCRIPTION

The exemplary embodiments described below are not intended to be exhaustive or to limit the invention to the precise forms disclosed in the following detailed description. Rather, the embodiments are chosen and described so that others skilled in the art may appreciate and understand the principles and practices of the present invention.

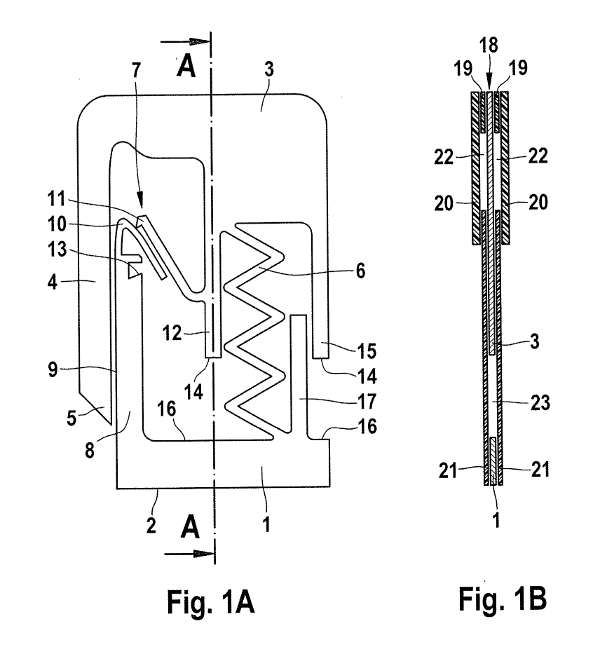

FIG. 1A is a schematic representation of the front view of a first embodiment of the pricking device.

The pricking device comprises a contact element 1 for placing into contact with an extraction surface (not shown), by applying its underside 2 to the extraction surface. The pricking device further comprises an actuating element 3 for manual actuation of the pricking device. A pricking element 4 with a tip 5 is fixedly connected to the actuating element. A return spring 6 is also provided for automatically retracting the pricking element after completion of a pricking procedure, and a locking mechanism 7 is provided for locking the pricking device after a single pricking operation has been completed.

The pricking device shown in FIG. 1A is shown in its unused state. The return spring 6 is in an unstressed state and the locking mechanism 7 is still unlocked. The pricking element 4 does not protrude past the underside 2 of the contact element 1.

A holding column 8 fixedly connected to the contact element 1 serves, inter alia, as one side of a guide 9 for the pricking element 4 in order to guide the pricking element 4 when it is being displaced relative to the contact element 1. The holding column 8 also supports a deflector 10 which functions as a switch and interacts with a catch hook 11, which functions as a bolt. The catch hook 11 is fixedly connected to the actuating element 3 by a catch hook support 12. The deflector 10 separates the catch hook 11 from a depression 13 in the holding column 8 (before actuation of the pricking device).

Moreover, the pricking device shown in FIG. 1A comprises a first limit stop 14 which is formed at the end of the catch hook support 12, along with a first limiting column 15 connected to the actuating element 3, and which interacts with a second limit stop 16 connected to the contact element 1. The first limiting column 15 also serves, together with the second limiting column 17, as a guide means during the pricking procedure.

FIG. 1B shows cross section A-A through the pricking device illustrated in FIG. 1A. The pricking device has a substantially flat design and is built into a plurality of layers. The inner part of the pricking device shown in FIG. 1A forms the inner layer 18, which is produced from a steel strip, and in particular, from surgical steel. In this embodiment, for example, the parts designated by reference numbers 1 to 17 are formed from the steel strip by micropunching, laser cutting, etching, or a combination thereof. The pricking elements 4 can then be ground, for which purpose they can be bent elastically upward or downward from the plane of the strip. The inner layer 18 is connected on both sides to film layers 19, 20, 21. These film layers are composed of plastic, and in an exemplary embodiment, of methyl methacrylate-butadiene-styrene (MBS). The film layers have a thickness between 50 μm to 2 mm, and more particularly between 125 μm to 500 μm.

FIG. 1B shows two cover films 21 secured to both sides of the contact element 1 and are flush with the lower edge of the contact element 1. They guide the inner layer 18 and its component parts and also partially cover them. The cover films 21 prevent the structural elements of the inner layer 18 from moving out of their planar orientation. Moreover, they cover the tip 5 of the pricking element 4, both in the unused state and used state of the pricking device, and thus form protection against touching said tip 5. Spacer films 19 are connected to the actuating element 3, and holding films 20 are fixed on these spacer films 19. The holding films 20 overlay one end of the cover films 21, but are not connected to them, with the result that they are able to slide over them. The holding films 20 serve as a grip for the pricking device. To improve the grip, other films can be applied to the actuating element 3. The spacer films 19 have the same thickness as the cover films 21 or are slightly thicker.

In an exemplary embodiment, the film layers 19, 20, 21 are secured by adhesive bonding. Films that are intended to move with the contact element 1 during actuation of the pricking device are secured only to the contact element 1, and those that are intended to move with the actuating element 3 are secured only to the actuating element 3. The chosen film thicknesses and the free spaces 22, 23, arranged between the individual film layers 19, 20, 21, ensure that the film layers 19, 20, 21 are able to move relative to one another with sufficient play and without being compressed. The film layers 19, 20, 21 can be fixed by contoured two-sided adhesive tapes, selectively applied hot-melt adhesives, reactive adhesives (in particular, epoxy adhesives), or UV-set adhesives (with sufficiently transparent films). To ensure a defined play, glass beads of defined size (for example with a diameter of 50 μm) can be added in a small amount to the adhesive.

Alternatively, the film layers 19, 20, 21 can also be fixed by thermal bonding. In this embodiment, at least two sufficiently large recesses are formed in the inner layer 18 (for example, in the steel strip). Thin, contoured spacer films 19 with a thickness between 10 μm to 50 μm, for example, are applied on both sides of the inner layer 18 and are covered by the thicker holding films 20. The assembly comprising films 19, 20 is welded in the area of the recesses in the inner layer 18 (in the steel strip). Heat is introduced using a hot die.

These pricking devices, being composed of a steel strip and film strips connected to both sides of the steel strip, can be produced continuously. After the assembly steps have been completed, the pricking devices are separated. In an exemplary method, this is done by punching, with a cut being punched through the plastic films and the steel strip. The steel strip is weakened in the area of the punch by at least one recess. In particular, only isolated connecting elements are present between two adjacent pricking devices and these can be separated by punching holes at the site of the connecting elements. This avoids the user being injured by protruding remnants of the steel strip. The films can be punched with a single, straight cut. Holding parts provided between the contact element and actuating element during production can also be removed in this separating operation.

After being separated, the pricking devices are placed singly or in groups in sterile packs and are then sterilized and packaged.

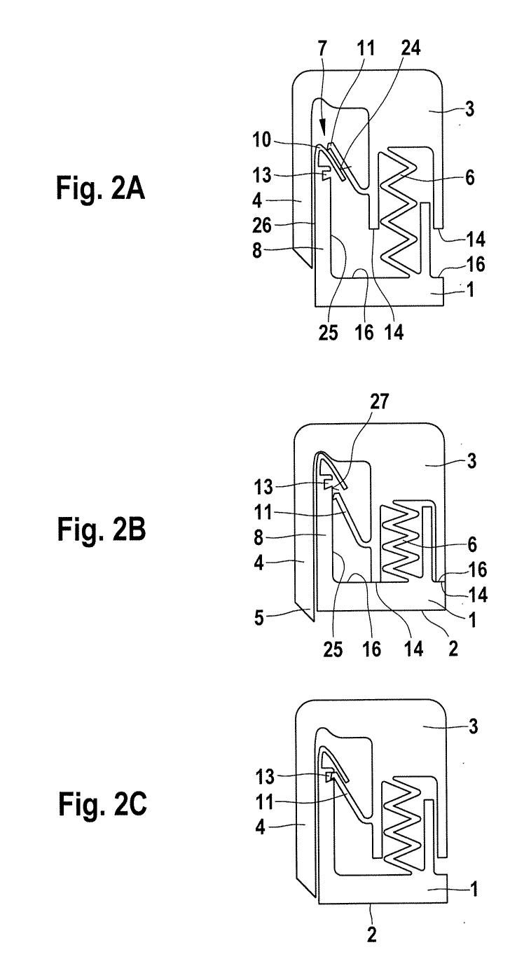

FIGS. 2A-2C show the actuation of the pricking device shown in FIGS. 1A and 1B. FIG. 2A shows the pricking device in the unused state. The return spring 6 is in an unstressed state and the locking mechanism 7 is unlocked. The catch hook 11, provided as a bolt, lies on the deflector 10, provided as a switch, and is separated from the depression 13 by said deflector 10. The surface of the deflector 10 on which the catch hook 11 lies is a first guide surface 24. The catch hook 11 slides along the first guide surface 24 upon initiation of the pricking procedure. A user of the device takes hold of the pricking device and presses it quickly against the extraction surface (not shown). The resistance offered by the extraction surface stops the movement of the contact element 1, whereas the actuating element 3 continues moving toward the extraction surface. At the same time, the return spring 6 is compressed and the catch hook 11 slides across the first guide surface 24 on the deflector 10. As the actuator continues to move, the catch hook 11 slides off the deflector 10 and then slides along the inside edge 25 of the holding column 8. Throughout this movement of the actuating element 3, the pricking element 4 is guided along the outside edge 26 of the holding column 8 toward the extraction surface. The relative movement of contact element 1 and actuating element 3 comes to a stop as soon as the first limit stop 14 strikes the second limit stop 16.

This state of the pricking device is shown in FIG. 2B. When the limit stops 14, 16 abut one another, the tip 5 of the pricking element 4 protrudes past the underside 2 of the contact element 1 and penetrates into the extraction surface. The pricking depth is therefore predetermined by the structure of the pricking device. The user's hand is stopped by the resistance offered by both the extraction surface to the pricking device and the abutment of both limit stops 14, 16. By the user moving the hand in the opposite direction, the pricking device is removed from the extraction surface. The return spring 6 pushes the contact element 1 and the actuating element 3 apart from one another. The catch hook 11 slides along the inside edge of the holding column 8, serving as the second guide surface 27, under the deflector 10 until it engages in the depression 13 in the holding column 8 and thus slides into the locked position. The locked position is shown in FIG. 2C. In this locked position, a reverse movement is no longer possible. Therefore, this prevents the pricking element 4 from being pushed out past the underside 2 of the contact element 1.

In the locked position, the actuating element 3 is arranged differently relative to the contact element 1 than it was before actuation of the pricking device. In FIG. 2C, the two elements are positioned closer together than they are in FIG. 2A. A usage indicator can communicate this change. In an exemplary embodiment, one of the films can be made of a transparent plastic such that the position of the catch hook 11 can be seen and the usage status can thus be determined visually. If non-transparent films are used, color markings or index holes can be provided in the cover films 21 (for example, index holes with a diameter of 0.5 to 0.6 mm) which, after the pricking device has been used, are closed by the actuating element 3 or covered by the holding films 20.

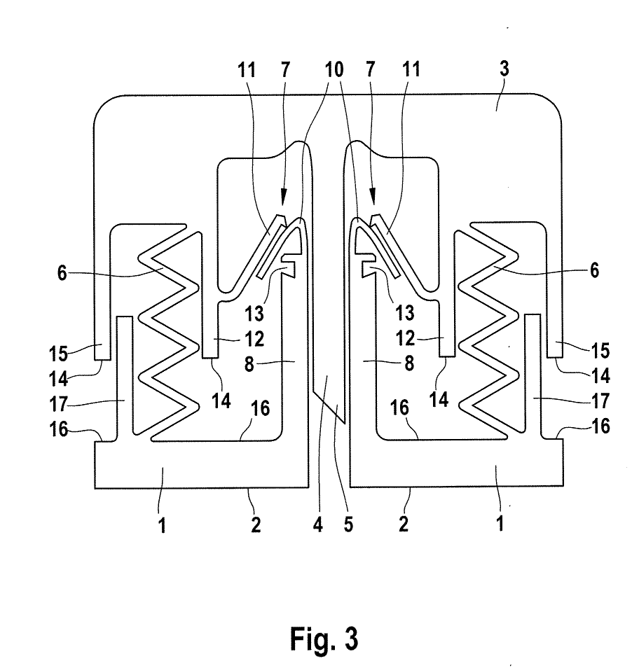

FIG. 3 shows a schematic representation of a second exemplary embodiment of a pricking device with two locking mechanisms. In this pricking device, two locking mechanisms 7 with identical actions are arranged symmetrically with respect to the pricking element 4. The locking mechanisms 7 each comprise a catch hook 11, a deflector 10, a depression 13, a holding column 8, and a catch hook support 12. Moreover, in this embodiment, two contact elements 1 and two return springs 6 are arranged symmetrically with respect to the pricking element 4 and the pricking device has one actuating element 3 engaging over these. The remaining elements of each half of the pricking device shown in FIG. 3 correspond to the elements of the pricking device shown in FIG. 1A and are designated by the same reference numbers. Compared to the pricking device shown in FIG. 1A, the pricking device shown in FIG. 3 has the advantage of ensuring more precise guiding of the pricking element 4. This pricking device can also be produced in the same way as that described above for the pricking device shown in FIG. 1A (for example, from a steel strip having film strips connected to it).

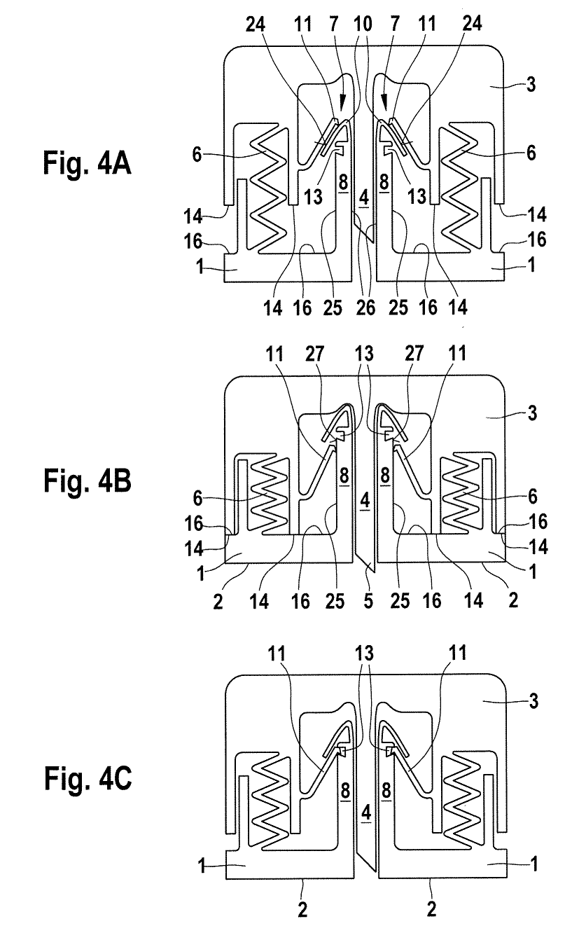

FIGS. 4A to 4C show the method involved in the actuation of a pricking device as shown in FIG. 3, with the start position being shown in FIG. 4A, the pricking position being shown in FIG. 4B, and the end position being shown in FIG. 4C. The method (and the corresponding reference numbers) is the same as that described for FIGS. 2A-2C, except that two return springs 6, initially in an unstressed state (FIG. 4A), are simultaneously compressed upon actuation (FIG. 4B) and two initially unlocked locking mechanisms (FIG. 4A), arranged symmetrically with respect to the pricking element 4, are both locked after the pricking procedure is completed (FIG. 4C).

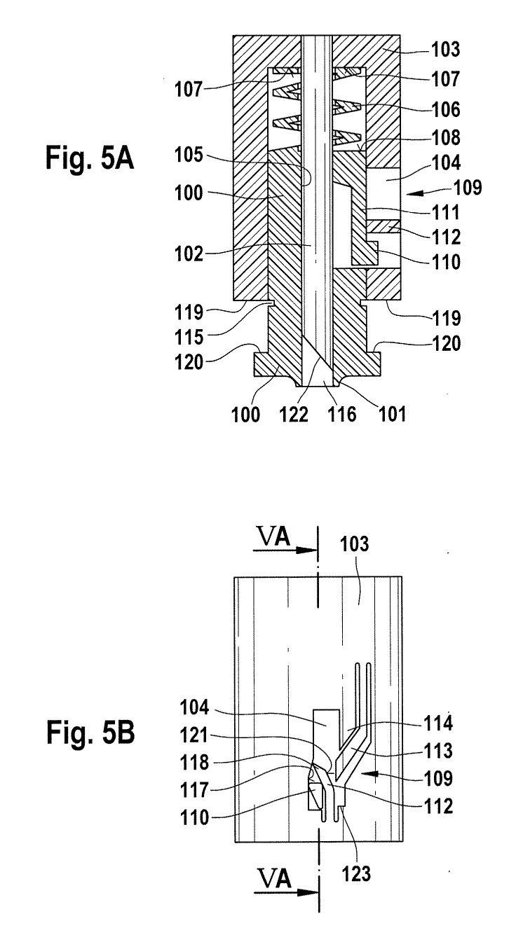

FIGS. 5A and 5B show a schematic representation of a third embodiment of a pricking device according to the invention. FIG. 5A shows a cross-section of line VA-VA in FIG. 5B through the pricking device and FIG. 5B shows a side view of the upper part of the pricking device from FIG. 5A from the outside.

The pricking device comprises a contact element 100 to be placed in contact with an extraction surface via a ring 101. The ring 101 tensions the skin before a pricking element 102 pierces it. The contact element 100 is arranged displaceably in an actuating element 103. The pricking element 102 is fixedly connected to the actuating element 103. The pricking element 102 can be made of metal, for example, and can be encapsulated by an actuating element 103 made of plastic. The pricking element 102 is preferably made from a conventional surgical-grade steel and with a diameter between 0.2-2.0 mm. Different embodiments of the ground edge 122 can be used. The actuating element 103 is a cylindrical sleeve which is closed at the top and has a lateral recess 104. The contact element 100 is a cylindrical sleeve with a central longitudinal bore 105. The actuating element 103 is in the form of a plastic sleeve closed at the top. A steel needle, for example, is fixed as the pricking element 102 in the closed lid of the actuating element 103. This fixing is done by encapsulation during production of the actuating element 103 in an injection molding operation. The internal diameter is such that the actuating element 103 can slide along the contact element 100 without friction. The insertion of the actuating element 103 into the contact element 100 is limited by a second limit stop 120 engaging a first limit stop 119. In an exemplary embodiment, the contact element 100 is provided as a plastic sleeve. The sleeve has, at its center, the longitudinal bore 105 with a diameter of 0.5-3 mm. The external diameter is approximately 3-10 mm. During use, the ring 101 at the lower end touches the skin. In the outer contour or wall, the sleeve has at least one limit stop 115 that limits the distance by which the actuating element 103 can be pushed onto the contact element 100.

The pricking device also comprises a return spring 106 for automatic retraction of the pricking element 102 after a pricking procedure has been completed, the return spring 106 being in an unstressed state before the pricking procedure is initiated. Measures for maintaining a tensioned position are therefore not needed. The return spring 106, designed as a helical spring, surrounds the pricking element 102 and rests on an inner, first annular surface 107 of the actuating element 103 and on an upper, second annular surface 108 of the contact element 100.

The pricking device further comprises a locking mechanism 109 for locking the pricking device after completion of a single pricking procedure, as is shown in FIG. 5B. The locking mechanism 109 comprises a bolt 110, designed as a guide block, which is fixedly connected to the contact element 100 via a spring element 111 and protrudes into the lateral recess 104 of the actuating element 103. For assembly purposes, this guide block 110 can be pressed inward by spring element 111. In its rest position, the guide block 110 also prevents easy separation or dismantling of the contact element 100 and actuating element 103. The locking mechanism 109 also includes a switch 112 which is designed such that the guide block 110 moves the switch 112 from a rest position upon actuation of the pricking device, and the switch 112 automatically moves back into the rest position after the guide block 110 has passed. The switch 112 is fixedly connected to the actuating element 103. The locking mechanism 109 further comprises a second switch 113, which can only be seen in FIG. 5B. The second switch 113 is fixedly connected to the actuating element 103. The guide block 110, which has passed the switch 112 during actuation of the pricking device, moves the second switch 113 from a starting position during a movement of the pricking device into the locked position, and the second switch 113 moves back automatically into the starting position after the guide block 110 has passed. Consequently, the guide block 110 is locked to the actuating element 103. To lock the guide block 110, the switch 113 bears on an abutment 114.

The contact element 100 has a circumferential groove 115 serving as a usage indicator. The groove 115 can be seen when the pricking device has not yet been used and is concealed by the actuating element 103 when the pricking device has been used and is in the locked position.

The actuation of the exemplary embodiments shown in FIG. 5A and FIG. 5B will now be explained. A user picks up the pricking device and presses it quickly against the extraction surface such as a patient's skin. When the ring 101 makes contact with the skin, the latter is tensioned across the opening 116 of the contact element 100, generally independently of the impact angle. The resistance offered by the extraction surface stops the forward movement of the contact element 100, whereas the actuating element 103 continues moving toward the direction of the extraction surface. In this way, the pricking element 102 is pushed into the skin as soon as it protrudes past the ring 101 of the contact element 100. At the same time, as a result of the actuating element 103 and contact element 100 being pushed together, the guide block 110 slides upward between the switch 112 and the edge of the recess 104 acting as first guide surface 117. In doing so, the guide block 110 forces the switch 112 sideways. After the guide block 110 has passed the switch 112, the latter snaps back into its rest position in which the tip 118 of the switch rests on the first guide surface 117. The actuating element 103 and the contact element 100 are pushed together until a first limit stop 119 contained in the actuating element 103 strikes a second limit stop 120 contained in the contact element 100. The depth to which the pricking element 102 pierces the extraction surface is defined by these limit stops 119, 120. When the limit stops 119, 120 strike one another, the user's hand senses a resistance. By the user moving the hand in the opposite direction, the pricking device is removed from the extraction surface. The return spring 106 pushes the contact element 100 back out of the actuating element 103. In this way, the pricking element 102 is again drawn back completely into the longitudinal bore 105. At the same time, on the other side of the switch 112, the guide block 110 slides down along a second guide surface 121 between the switch 112 and the second switch 113. In doing so, the guide block 110 forces the second switch 113 sideways. After the guide block 110 has passed the second switch 113, the latter springs back into its rest position on the abutment 114. The guide block 110 is stopped by a shoulder 123. In this locked position, the contact element 100 and the actuating element 103 are not pushed as far apart as in their starting position. Accordingly, the groove 115, acting as usage indicator, remains concealed by the actuating element 103.

Repeated use of the pricking device is prevented because the second switch 113 blocks an upward movement of the guide block 110 and thus prevents the contact element 100 and the actuating element 103 from being pushed together. The switch 113 cannot deflect upward, because it is pressed onto the abutment 114. A re-emergence of the pricking element 102 through the opening 116 is thus blocked.

Instead of being in the form of a groove 115, a usage indicator can also be designed as two index holes which no longer lie in alignment with one another after the pricking device has been used once. A further possibility includes the provision of color markings on the contact element 100 and/or on the actuating element 103, by which the displacement can be visually identified after use.

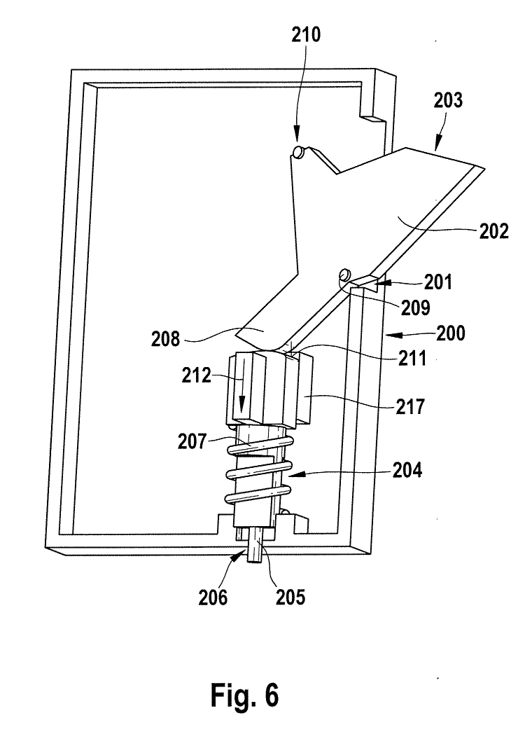

FIG. 6 shows a schematic representation of a fourth exemplary embodiment of a pricking device with an actuating element designed as a lever that can turn about a hinge pin. The pricking device comprises a contact element 200 to be placed in contact with an extraction surface (not shown). The contact element 200 is a housing with a recess 201. The housing 200 is shown open in FIG. 6, but generally is closed from above by a second housing half which can, for example, be welded onto the illustrated housing half or be plugged onto it and mechanically connected. The actuating element is in the form of a lever 202 that can turn about a hinge pin 209. Before the pricking device is actuated, a proximal end 203 of the lever 202 protrudes through the recess 201. Arranged in the contact element 200, and displaceable relative to the contact element 200 in a direction substantially perpendicular to the extraction surface and along a guide 217, there is a pricking element 204 whose tip 205 is oriented toward an opening 206 in the contact element 200. A return spring 207 is provided for automatic retraction of the pricking element 204 into the contact element after a pricking procedure is completed, the return spring 207 being in an unstressed state before the pricking procedure is initiated. The return spring 207 can be made of steel, for example, or can be injection molded from plastic. A locking mechanism provided in the pricking device comprises a bolt 208, which is provided in the form of distal end 208 of the lever 202, that can turn about the hinge pin 209. The hinge pin 209 of the lever 202 is fixedly connected to the housing 200. The lever 202 is secured by a holding element 210 before the pricking device is actuated, with a release force being needed in order to release the lever 202. The holding element 210 is designed such that it breaks or buckles, or frees the lever 202 in some other way, when the release force is exceeded. Before the pricking device is actuated, the lever 202 lies with its distal end 208 on the contact surface 211 of the pricking element 204, as shown in FIG. 6. Upon actuation of the pricking device, the distal end 208 of the lever 202, through manual actuation of its proximal end 203, applies a force to the pricking element 204 along the curved contact surface 211 in the pricking direction 212. The pricking device can be provided with a sterile protection and/or can be placed in a sterile package.

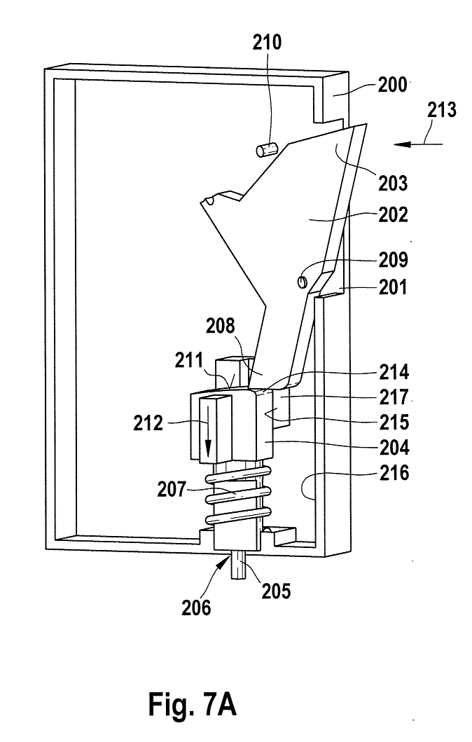

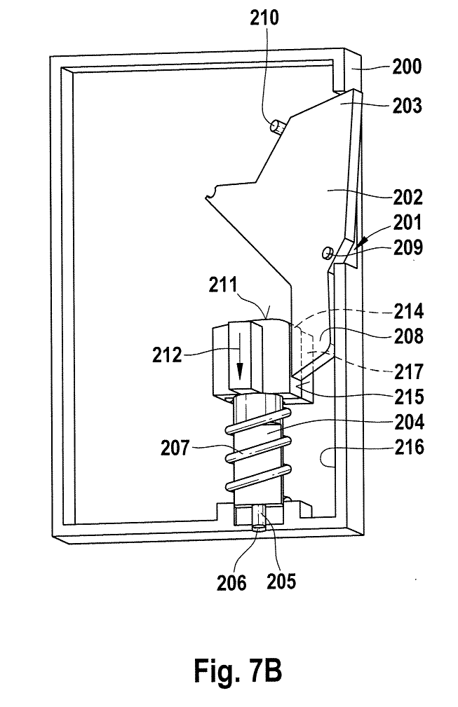

FIGS. 7A and 7B show the method involved in the actuation of the pricking device shown in FIG. 6. To actuate the pricking device, the opening 206 in the contact element 200 is placed onto an extraction surface (not shown), and a user applies a force directly against the proximal end 203 of the lever 202 in actuation direction 213. When the release force is reached, the holding element 210 frees the lever 202 by buckling, breaking or yielding of the holding element 210 to turn about the hinge pin 209. The release force then accelerates the lever, which turns about the hinge pin 209, and with its distal end 208, exerts a force in pricking direction 212 on the contact surface 211 of the pricking element 204. The pricking element 204 thus suddenly moves along the guide 217 in the direction of the extraction surface. At the same time, the return spring 207 is expanded. The contact surface 211 is delimited by an edge 214. The distal end 208 of the lever 202 slides across the inclined contact surface 211, or first guide surface 211, to the edge 214 upon actuation of the proximal end 203. As soon as the distal end 208 of the lever 202 has passed the edge 214 of the contact surface 211, the pricking element 204 is retracted by the return spring 207 completely into the housing 200 as the distal end 208 no longer applies a force to the pricking element 204 in the pricking direction 212. During the retraction of the pricking element 204, the distal end 208 of the lever 202, serving as a bolt, slides along the second guide surface 215, arranged laterally on the pricking element 204, and into a locked position (shown in FIG. 7B). Therefore, the pricking element 204 is designed with two guide surfaces 211, 215 and with the edge 214 serving as a switch.

In the locked position, the distal end 208 of the lever 202 is fixed between the inside wall 216 of the housing 200 and the second guide surface 215. Additionally, the pricking element 204 has retracted completely into the housing 200 by the return spring 207 and thereby the lever 202 cannot turn about the hinge pin 209. The proximal end 203 of the lever 202 is disposed substantially into the housing 200 in the locked position. This can be visually observed and can serve as a usage indicator. Repeated use of the pricking device is now impossible.

In this exemplary embodiment, the pricking depth is fixed by the relative arrangement of lever 202, pricking element 204, and housing 200.

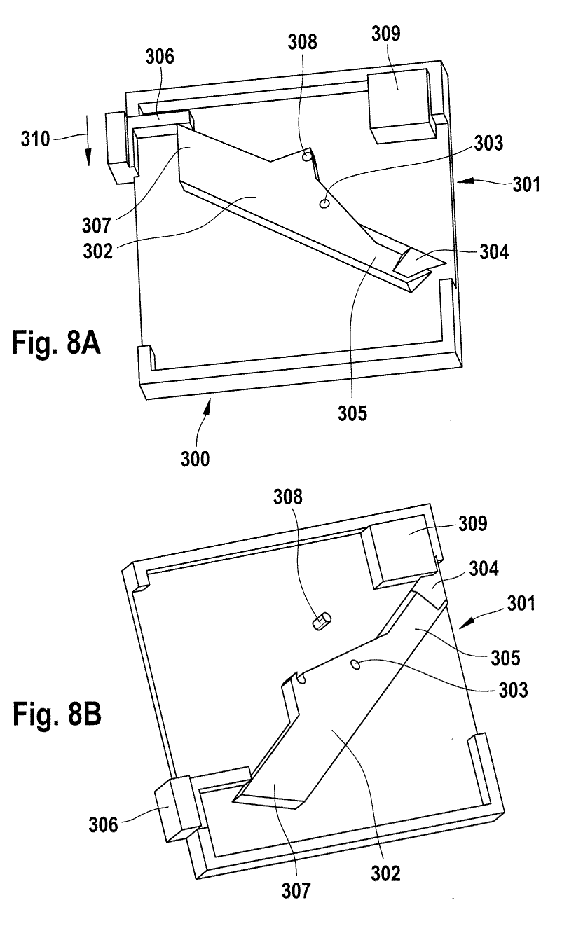

FIGS. 8A and 8B show a schematic representation of a fifth exemplary embodiment of a pricking device with a rotatable lever and a blade for perforating the extraction surface. The pricking device comprises a contact element 300 to be placed in contact with an extraction surface. The contact element 300 has an opening 301. An actuating element 302, designed as a lever 302 that can turn about a hinge pin 303, is arranged in the contact element 300, which is designed as a housing 300. The housing 300 is shown open in FIGS. 8A and 8B, but is in fact closed from above by a second housing half which is welded onto the illustrated housing half or plugged onto it and mechanically connected. The lever 302 interacts with the pricking element 304 in order to perforate the extraction surface, the pricking element 304 in this embodiment being a blade 304 that is fixedly connected to the distal end 305 of the lever 302. A force for actuating the pricking device can be applied indirectly to the proximal end 307 of the lever 302 via button 306.

The pricking device further comprises a holding element 308 by which the lever 302 is secured before actuation of the pricking device. A release force is needed in order to release the lever 302 and when the release force is exceeded, the holding element 308 breaks or buckles, or in some other way frees the lever 302 to turn about the hinge pin 303.

In this embodiment of the pricking device, the locking mechanism comprises a block element 309 into which the blade 304 enters after completion of a pricking procedure, such that it is fixed therein and the lever 302 is thereby locked.

This embodiment does not require a return spring and it is therefore possible to dispense with one structural part and reduce costs. With the aid of the lever 302 that can turn about the hinge pin 303 and the blade 304 that is secured thereon, the pricking device executes a guided cutting movement.

To actuate the pricking device, the opening 301 in the contact element 300 is pressed onto an extraction surface. The user applies a force to the button 306 in the actuation direction 310, which force thus acts indirectly on the proximal end 307 of the lever 302. When the release force is reached, the holding element 308 frees the lever 302 to turn about the hinge pin 303, for example, by buckling, breaking or yielding of the holding element 308. The release force then accelerates the lever 302, which thereby executes a sudden rotational movement about the hinge pin 303. The blade 314 executes a cutting movement in a circular trajectory through the opening 301 and thus perforates the extraction surface. The pricking depth is defined by the arrangement of the lever 302 relative to the opening 301 in the housing 300. As soon as the blade 304 reaches the block element 309, it cuts its way into the material of the block element 309, such that it is fixed therein and the lever 302 has reached its locked position.

The pricking device can be provided with a sterile protection and/or can be placed in a sterile package.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE NUMERAL DESIGNATIONS

1 contact element
2 underside of contact element
3 actuating element
4 pricking element
5 tip of pricking element
6 return spring
7 locking mechanism
8 holding column
9 guide
10 deflector=switch
11 catch hook=bolt
12 catch hook support
13 depression
14 first limit stop
15 first limiting column
16 second limit stop
17 second limiting column
18 inner layer
19 spacer films
20 holding films
21 cover films
22 first free space
23 second free space
24 first guide surface
25 inner edge of holding column
26 outer edge of holding column
27 second guide surface
100 contact element
101 ring
102 pricking element
103 actuating element
104 recess
105 longitudinal bore
106 return spring
107 first annular surface
108 second annular surface
109 locking mechanism
110 bolt=guide block
111 spring element
112 switch
113 second switch
114 abutment
115 usage indicator=groove
116 opening
117 first guide surface
118 tip of switch
119 first limit stop
120 second limit stop
121 second guide surface
122 ground edge
123 shoulder
200 contact element=housing
201 recess
202 actuating element=lever
203 proximal end
204 pricking element
205 tip
206 opening
207 return spring
208 distal end=bolt
209 hinge pin
210 holding element
211 contact surface=first guide surface
212 pricking direction
213 actuating direction
214 edge
215 second guide surface
216 inside wall
217 guide
300 contact element=housing
301 opening
302 actuating element=lever
303 hinge pin
304 pricking element
305 distal end
306 button
307 proximal end
308 holding element
309 block element
310 actuating direction

What is claimed is:

1. A pricking device for withdrawing blood, comprising:
a contact element configured for contacting an extraction surface;
an actuating element configured for manually actuating the pricking device;
a pricking element which is displaceable relative to the contact element in a direction substantially perpendicular to the extraction surface, the pricking element being arranged in the contact element such that a defined pricking depth is obtained during the pricking procedure, the pricking element being positioned within the contact element before the pricking procedure is initiated;
a return spring configured to automatically retract the pricking element into the contact element after the pricking procedure has been completed; and
a locking mechanism configured to lock the pricking device after completion of a single pricking procedure, the locking mechanism comprising a bolt defined by one of the contact element and the actuating element and a switch defined by the other of the contact element and the actuating element, the bolt configured to slide along a first guide surface of the switch when the pricking procedure is initiated and then slide along a second guide surface of the switch when the pricking element is retracted by the return spring after completion of the pricking procedure.

2. The pricking device of claim 1, wherein the return spring is configured in a substantially unstressed state before the pricking procedure is initiated.

3. The pricking device of claim 1, further comprising a usage indicator for displaying whether the pricking device has been used.

4. The pricking device of claim 3, wherein the usage indicator is based upon the actuating element being arranged relative to the contact element differently before the pricking device is actuated than after the pricking device has been locked by the locking mechanism.

5. The pricking device of claim 1, wherein the pricking element comprises a needle, a lancet or a blade.

6. The pricking device of claim 1, further comprising a guide for guiding the pricking element while it is being displaced relative to the contact element.

7. The pricking device of claim 1, wherein a compression or an expansion of the return spring takes place during the pricking procedure.

8. The pricking device of claim 1, wherein the actuating element comprises a push-button with a push surface.

9. The pricking device of claim 1, wherein the locking mechanism comprises two locking mechanisms with identical actions arranged symmetrically with respect to the pricking element.

10. The pricking device of claim 9, wherein the contact element comprises two contact elements arranged symmetrically with respect to the pricking element, and the return spring comprises two return springs arranged symmetrically with respect to the pricking element.

11. The pricking device of claim 1, wherein the contact element is displaceably mounted in the actuating element or the actuating element is displaceably mounted in the contact element.

12. The pricking device of claim 1, wherein the pricking element is fixedly connected to the actuating element.

13. The pricking device of claim 12, wherein the pricking element is metal and is encapsulated by the actuating element, the actuating element being made of plastic.

14. The pricking device of claim 1, wherein the bolt is integrally formed with and extends from the contact element or the actuating element.

15. The pricking device of claim 1, wherein the bolt in the locked position engages a projection or a depression.

16. The pricking device of claim 1, further comprising a first limit stop contained in the actuating element and a second limit stop contained in the contact element, wherein the first and second limit stops interact when the defined pricking depth is reached.

17. The pricking device of claim 1, wherein the switch comprises a deflector fixedly connected to the contact element and the bolt comprises a hook fixedly connected to the actuating element.

18. The pricking device of claim 17, wherein the deflector is configured such that, before actuation and during displacement of the pricking element toward the extraction surface, the deflector maintains the hook spaced away from a depression in the contact element and guides the hook along the first guide surface, further wherein, after a pricking procedure has been completed, the hook is guided by the second guide surface into the depression, whereby the hook is then locked in the depression in the locked position.

19. The pricking device of claim 1, wherein the pricking device has a substantially flat design and is comprised of multiple layers.

20. The pricking device of claim 19, wherein the contact element, the actuating element, the pricking element, the return spring and the locking mechanism are all formed from a unitary metal strip.

21. The pricking device of claim 20, wherein the metal strip has a width of 10 mm to 50 mm and a thickness of 0.1 mm to 2 mm.

22. The pricking device of claim 20, wherein the metal strip is connected on both sides to at least one film strip.

23. The pricking device of claim 22, wherein the metal strip is connected on both sides to a plurality of mutually displaceable film strips.

24. The pricking device of claim 22, wherein the at least one film strip has a thickness of 50 μm to 2 mm.

25. The pricking device of claim 22, wherein the at least one film strip is connected to the metal strip by an adhesive.

26. The pricking device of claim 25, wherein the adhesive contains glass beads.

27. The pricking device of claim 22, wherein the at least one film strip is transparent.

28. The pricking device of claim 1, wherein the actuating element comprises a cylindrical sleeve which is closed at the top and which includes at least one lateral recess.

29. The pricking device of claim 28, wherein the contact element comprises a cylindrical member having a central longitudinal bore.

30. The pricking device of claim 28, wherein the return spring comprises a helical spring surrounding the pricking element, the return spring being supported on an inner, first annular surface of the actuating element and on an upper, second annular surface of the contact element.

31. The pricking device of claim 28, wherein the bolt comprises at least one guide block which is fixedly connected to the contact element by a spring element and which protrudes into the lateral recess of the actuating element.

32. The pricking device of claim 31, wherein the switch is fixedly connected to the actuating element and configured such that the guide block moves the switch from a rest position upon actuation of the pricking device, and the switch automatically moves back into the rest position after the guide block has passed the switch.

33. The pricking device of claim 32, further comprising a second switch fixedly connected to the actuating element, the guide block moving the second switch from a starting position during a movement of the pricking device and the second switch automatically returning to its starting position after the guide block has passed, wherein a locking of the guide block to the actuating element is achieved.

34. The pricking device as claimed in claim 33, wherein the second switch bears on an abutment.

35. A pricking device for withdrawing blood, comprising:
a contact element configured for contact with an extraction surface;
a pricking element for perforating the extraction surface; and
an actuating element configured for manually actuating the pricking device, the actuating element comprising a lever configured to turn about a hinge pin to move the pricking element; and
a holding element which secures the lever before actuation of the pricking device, wherein the lever requires a release force applied thereto to disengage the lever from the holding element.

36. The pricking device of claim 35, wherein the holding element is configured to break or buckle to disengage the lever when a force equal to or exceeding the release force is applied to the lever.

37. The pricking device of claim 35, wherein the lever comprises a distal end which engages the pricking element and a proximal end which is manually actuated.

38. The pricking device of claim 37, wherein, before the pricking device is actuated, the distal end of the lever rests on a contact surface of the pricking element, the pricking element being displaceable in a direction substantially perpendicular to the extraction surface, wherein manual actuation of the proximal end of the lever applies a force to the pricking element via the contact surface.

39. The pricking device of claim 37, wherein the distal end of the lever is fixedly connected to the pricking element, the pricking element comprising a blade.

40. The pricking device of claim 35, further comprising a locking mechanism for locking the pricking device after completion of a pricking procedure, the locking mechanism locking the lever in an end position.

41. The pricking device of claim 35, wherein the contact element comprises a housing with a recess, wherein a proximal end of the lever protrudes through the recess before the pricking device is actuated.

42. The pricking device of claim 41, wherein the proximal end of the lever retracts into the housing when the pricking device is actuated.

43. The pricking device of claim 35, wherein:
the pricking element comprises a return spring and a contact surface terminating in an edge;
the lever comprises a distal end configured to slide across the contact surface and a proximal end configured for manual actuation;
the contact element comprises a housing which houses the pricking element and the lever; and
further wherein, upon actuation of the proximal end of the lever, the distal end of the lever slides across the contact surface and advances the pricking element in a pricking direction until the distal end of the lever passes the edge, whereafter the return spring draws the pricking element completely into the housing and the distal end of the lever is positioned in a locked position in which it no longer applies a force to the pricking element in the pricking direction.

44. The pricking device of claim 43, wherein the distal end of the lever, in the locked position, is fixed between an inside wall of the housing and the pricking element.

45. The pricking device of claim 43, wherein the proximal end is positioned substantially within the housing in the locked position.

46. A method for producing a pricking device of the type comprising a contact element for contacting an extraction surface, an actuating element for actuating the pricking device, a displaceable pricking element, and a return spring configured to automatically retract the pricking element into the contact element after the pricking procedure has been completed, the method comprising forming the contact element, the actuating element, the pricking element, the return spring and the locking mechanism from a metal strip by micro-punching, laser cutting, etching, or a combination thereof.

47. The method of claim 46, further comprising forming the contact element and the actuating element integrally, wherein the contact element and actuating element are initially connected by a holding part, and then removing the holding part after the contact element and actuating element have been formed.

48. The method of claim 46, further comprising forming a plurality of contiguous pricking devices from the metal strip.

49. The method of claim 48, further comprising separating the individual pricking devices from one another by punching through connecting elements provided between them.

50. The method of claim 48, further comprising adhering a film strip to the single metal strip by adhesive or thermal bonding.

51. The method of claim 50, further comprising severing the film strip together with the metal strip by punching with straight cuts.

52. The method of claim 50, further comprising arranging two film strips on both sides of the metal strip, and welding the films over a recess in the metal strip.

53. A pricking device for withdrawing blood, comprising:
a contact element configured for contacting an extraction surface;
an actuating element configured for manually actuating the pricking device;
a pricking element which is displaceable relative to the contact element;
a return spring configured to automatically retract the pricking element into the contact element after the pricking procedure has been completed; and
wherein the contact element, the actuating element, the pricking element, and the return spring are formed from a single metal strip having a severed holding element that separates the contact element from the actuating element.

54. The pricking device of claim 53, further comprising a locking mechanism configured to lock the pricking device after completion of a single pricking procedure, the locking mechanism comprising a bolt and a switch, the bolt configured to slide along a first guide surface of the switch when the pricking procedure is initiated and then slide along a second guide surface of the switch when the pricking element is retracted by the return spring after completion of the pricking procedure.

55. The pricking device of claim 53, further comprising a first limit stop contained in the actuating element and a second limit stop contained in the contact element, wherein the first and second limit stops interact when the defined pricking depth is reached.

56. The pricking device of claim 53, wherein the metal strip is connected on both sides to at least one film strip.

57. The pricking device of claim 56, wherein the metal strip is connected on both sides to a plurality of mutually displaceable film strips.

58. The pricking device of claim 56, wherein the at least one film strip is connected to the metal strip by an adhesive containing beads.

59. The pricking device of claim 56, wherein the at least one film strip is transparent.

60. A pricking device for withdrawing blood, comprising:
a contact element configured for contacting an extraction surface;
an actuating element configured for manually actuating the pricking device;
a pricking element which is displaceable relative to the contact element;
a return spring configured to automatically retract the pricking element into the contact element after the pricking procedure has been completed; and
the contact element, the actuating element, the pricking element, and the return spring together forming an inner layer having opposite sides, each side having two films thereon that are displaceable relative to one another when the pricking device is actuated.

61. The pricking device of claim 60, wherein the two films comprise a cover film that is secured to the inner layer and a holding film that is displaceable relative to the cover film when the pricking device is actuated.

62. The pricking device of claim 61, further comprising a spacer film disposed on each side of the inner layer, each spacer film having one side adhered to the inner layer and its other side adhered to one of the holding films.

63. The pricking device of claim 62, wherein, on each side of the inner layer, the spacer film and the cover film are arranged in substantially the same plane.

64. The pricking device of claim 62, wherein the cover films maintain the contact element, actuating element, pricking element and return spring in a substantially planar orientation during use of the pricking device.

65. The pricking device of claim 62, further comprising a locking mechanism configured to lock the pricking device after completion of a single pricking procedure, the locking mechanism comprising a bolt and a switch, the bolt configured to slide along a first guide surface of the switch when the pricking procedure is initiated and then slide along a second guide surface of the switch when the pricking element is retracted by the return spring after completion of the pricking procedure.

66. The pricking device of claim 62, wherein at least one of the cover films is at least partially transparent.

67. The pricking device of claim 62, wherein the spacer film is adhered to the holding film and to the inner layer by an adhesive.

68. The pricking device of claim 62, wherein the inner layer comprises at least one recess on each side thereof, wherein the cover films or the spacer films are welded into the recesses.
